(19) United States
(12) Patent Application Publication
(10) Pub. No.: US 2016/0120472 A1
(43) Pub. Date: May 5, 2016

(54) Low Dissolution Rate Device and Method

(71) Applicant: The Government of the United States of America, as represented by the Secretary of the Navy, Washington, DC (US)

(72) Inventors: Francis J. Kub, Arnold, MD (US); Charles R. Eddy, JR., Columbia, MD (US); Virginia D. Wheeler, Alexandria, VA (US)

(21) Appl. No.: 14/928,318

(22) Filed: Oct. 30, 2015

Related U.S. Application Data
(60) Provisional application No. 62/073,414, filed on Oct. 31, 2014.

(51) Int. Cl. A61B 5/00; C23C 16/455

(52) U.S. Cl. CPC A61B 5/686; C23C 16/45525

(57) ABSTRACT

An implantable device includes a circuit protected with a low dissolution rate layer, wherein the circuit is either (a) fully encapsulated by the low dissolution rate layer and configured for non-electrical conduction contact sensing (e.g., capacitive sensing) or (b) partially encapsulated by the low dissolution rate layer with an electrode at least partially exposed outside the layer; wherein the implantable device is suitable for implantation inside the body of a living animal; and wherein the low dissolution rate layer comprises an element selected from the group consisting of gallium, boron, nitrogen, oxygen, zirconium, aluminum, and titanium. Such devices can be made by lithographic and other means, with coating layers applied by atomic layer deposition.

### CROSS-REFERENCE TO RELATED APPLICATIONS

This application claims the benefit of U.S. Provisional Application 62/073,414 filed on Oct. 31, 2014, the entirety of which is incorporated herein by reference.

### BACKGROUND

Electrodes devices that are exposed to a harsh environment (for example, those implanted into a tissue or otherwise exposed to a physiological environment) can chemically react with the environment and the materials used to form the implantable electrode can be etched or dissolved by the harsh environment. A need exists to mitigate such reactions.

### BRIEF SUMMARY

In one embodiment, an implantable device includes a circuit protected with a low dissolution rate layer, wherein the circuit is either (a) fully encapsulated by the low dissolution rate layer and configured to perform non-electrical conduction contact sensing, or (b) partially encapsulated by the low dissolution rate layer with an electrode at least partially exposed outside the layer; wherein the implantable device is suitable for implantation inside the body of a living animal; and wherein the low dissolution rate layer comprises at least one element selected from the group consisting of gallium, boron, nitrogen, oxygen, zirconium, aluminum, and titanium.

In another embodiment an implantable device includes a circuit protected with an inner insulation layer in a state of having been deposited by atomic layer deposition which in turn is surrounded by and in intimate contact with an outer low dissolution rate layer, wherein the circuit is either (a) fully encapsulated by both layers and configured to perform non-electrical conduction contact sensing, or (b) partially encapsulated by both layers with only an electrode at least partially exposed outside the layers; wherein the implantable device is suitable for implantation inside the body of a living animal; and wherein the low dissolution rate layer is in a state of having been deposited by atomic layer deposition and comprises a material selected from the group consisting of gallium, boron, nitride, oxide, zirconium, aluminum, titanium, gallium nitride, boron nitride, zirconium oxide, zirconia oxide, diamond, aluminum oxide, titanium nitride, titanium carbide, titanium dioxide, and combinations thereof.

An additional embodiment is a method of making a low dissolution rate device, the method including providing a substrate; coating via atomic layer deposition a first low dissolution layer comprising at least one an element selected from the group consisting of gallium, boron, nitrogen, oxygen, zirconium, aluminum, and titanium, constructing a circuit on the substrate; and coating the circuit via atomic layer deposition a second low dissolution layer comprising at least one an element selected from the group consisting of gallium, boron, nitrogen, oxygen, zirconium, aluminum, and titanium, thereby obtaining an implantable device comprising a circuit protected with a low dissolution rate layer, wherein the circuit is either (a) fully encapsulated by the low dissolution rate layer and configured to perform non-electrical conduction contact sensing, or (b) partially encapsulated by the low dissolution rate layer with only an electrode at least partially exposed outside the layer; wherein the implantable device is suitable for implantation inside the body of a living animal.

A further embodiment is a method of making a low dissolution rate device, the method including providing a substrate; constructing a circuit on the substrate; and coating, via atomic layer deposition, the circuit with a low dissolution rate layer comprising at least one an element selected from the group consisting of gallium, boron, nitride, oxide, zirconium, aluminum, and titanium thereby obtaining an implantable device comprising a circuit protected with a low dissolution rate layer, wherein the circuit is either (a) fully encapsulated by the low dissolution rate layer and configured to perform non-electrical conducting contact sensing, or (b) partially encapsulated by the low dissolution rate layer with an electrode at least partially exposed outside the layer; wherein the implantable device is suitable for implantation inside the body of a living animal.

Yet another embodiment is an additional method of making a low dissolution rate device, the method including providing a substrate, providing a release layer on a substrate, depositing a first coating material layer comprising at least one low dissolution rate material on the release layer, constructing on the first coating material layer a circuit comprising an electrode material, depositing on the circuit a second coating material layer comprising at least one low dissolution rate material such that both coating material layers contact each other at lateral sides of the circuit, depositing a strengthening material layer on the second coating material layer, etching a via through the strengthening material layer to the electrode material, and etching the release layer to release the implantable device; wherein the implantable device comprises the circuit protected with the low dissolution rate material, wherein the circuit is either (a) fully encapsulated by the low dissolution rate material and configured to perform non-electrical conduction contact sensing, or (b) partially encapsulated by the low dissolution rate layer with an electrode at least partially exposed outside the layer; wherein the implantable device is suitable for implantation inside the body of a living animal; and wherein the low dissolution rate material comprises at least one element selected from the group consisting of gallium, boron, nitrogen, oxygen, zirconium, aluminum, and titanium.

### BRIEF DESCRIPTION OF THE DRAWINGS

FIG. 1 shows one exemplary embodiment of an flexible implantable device with a first coating material layer, a second coating material layer, and a topside polymer strengthening material layer according to the present invention.

FIG. 2 shows an exemplary embodiment of an flexible implantable device with a first coating material layer, a second coating material layer, and a topside polymer strengthening material layer with example material layers according to the present invention.

FIG. 3 shows an exemplary embodiment of an flexible implantable device with a first coating material layer, a second coating material layer, a topside polymer strengthening material layer, and a laterally etched electrical conductive low dissolution material layer according to the present invention.

FIG. 4 shows an exemplary embodiment of an flexible implantable device with a first coating material layer, a second coating material layer, a topside polymer strengthening material layer according to the present invention, wherein the first coating layer includes separate layers providing electrical insulation and low dissolution.

FIG. 5 shows one exemplary embodiment of an flexible implantable device with a first coating material layer, a second coating material layer, a topside polymer strengthening material layer, an optional anti-inflammatory material layer, and an optional dissolvable material layer according to the present invention.

FIG. 6 an exemplary embodiment of implantable device having MOSFET formed in a silicon-on-insulator substrate according to the present invention.

FIG. 7 shows an embodiment with a coating applied by atomic layer deposition (ALD) applied to device, the ALD coating having been deposited on the surface of a parylene polymer.

FIG. 8 shows an embodiment with a first ALD coating low dissolution layer on the parylene substrate underneath the electrical circuit and a second ALD coating low dissolution layer above the electrical circuit, with the ALD having been deposited throughout the surface of the electrical conductor material including wire bonds.

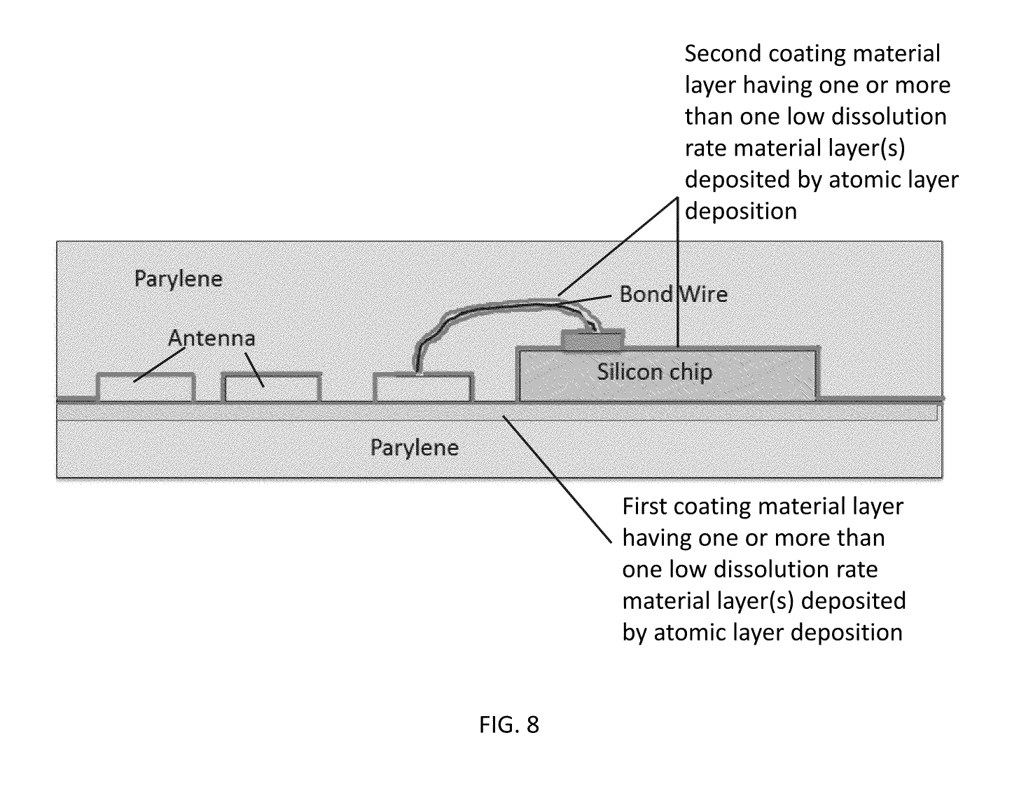

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

The term “low dissolution rate layer” as used herein refers to a layer of material having a low rate for dissolving, etching, or wearing away of a material layer by a chemical or electrical reaction when in a harsh environment, namely (unless otherwise specified), the environment experienced when implanted in a physiological environment. The term “low dissolution rate material” refers to a material effective to produce such a layer.

The term “substrate” as used herein may refer to either an inorganic substrate, i.e. a semiconductor substrate, or to a polymer substrate.

### Overview

Implantable devices, particularly such devices having electrodes, can be protected with a low dissolution rate layer. Such devices are termed low dissolution rate devices.

### Description and Operation

A device incorporating a low dissolution rate layer can take many forms—it may include a pressure sensor (for example, an ocular pressure sensor, a blood pressure sensor, an intra brain pressure sensor, or a bladder pressure sensor), other forms of sensor (for example, a pH sensor, a strain sensor, a temperature sensor), a pacemaker, a vagus nerve stimulator, a brain activity sensor, a deep brain stimulator, a heart stimulator, a bladder stimulator, a cochlear implant, a retina implant, a wireless transmitter, a wireless receiver, a transmitter and/or receiver for power transfer, and combinations of these.

Embodiments of such devices include one or more electrodes, one or more non-electrical conducting contact sensing devices, or one or more electrodes and one or more non-electrical conducting contact sensing device while further embodiments include methods of making them.

Non-electrical conduction contact sensing may include, for example, capacitive sensing, EKG sensing, strain sensing, pressure sensing (for example, an ocular pressure sensor, a blood pressure sensor, an intra-brain pressure sensor, or a bladder pressure sensor).

One embodiment is an implantable device having an electrode, termed an electrode device. An electrode device may comprise a sensor and/or a stimulating device that causes a current to be applied, such as a pacemaker. An electrode device can include an electrical conductor layer, electrode material, and one or more coating material layer(s). Such a device can further include a first coating material layer on the bottom side of the electrical conductor layer, a second coating material layer on the top side and lateral sides of the of the electrical conductor layer, and/or electrode material in contact with the electrical conductor that is exposed to the outer surface of the electrode device. Optionally, the electrode may be the only portion of the device exposed outside the coating.

A coating layer may include one or more layers of material, such as in a laminate, and preferably includes one or more low dissolution rate layer(s). The low dissolution rate layer(s) may be an electrical insulation layer(s) that provides electrical insulation of the electrical conductor layer(s) from the physiological environment for the portion of the electrode device that is adjacent to the physiological environment, e.g. in a tissue. Alternately, the low dissolution rate layer(s) can be electrically conducting. One or more electrical insulation layer(s) may be provided separately from the low dissolution rate layer(s).

A titanium nitride layer is an example of a low dissolution electrical conducting layer. One or more electrical insulation layer(s) may be provided between the electrical conductor layer and the titanium nitride low dissolution layer. An additional insulating low dissolution layer may be provided between the outer surface of the titanium nitride low dissolution electrical conducting layer and the electrode material to insulate the electrode from the titanium nitride low dissolution layer. The electrode material may overlap the outer electrical insulating layer to increase the dissolution time for the harsh environment to laterally dissolve the outer electrical insulating layer in the region of the opening in the outer electrical insulating layer for the electrode material to contact the electrical conductor. The overlap of the electrode material of the outer insulating low dissolution layer may be in the range of 0.1 microns to about 20 microns, for example.

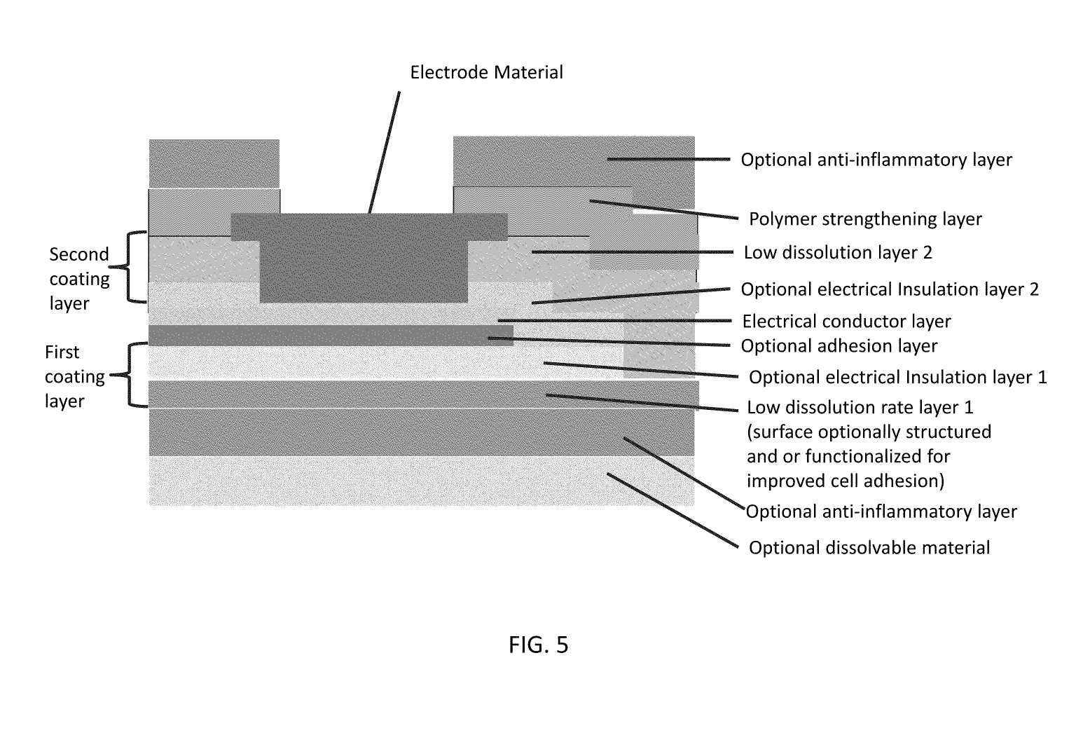

One or more layers of the coating material may have low corrosion rate in physiological environment. One or more of the material layers may be an adhesion layer for the electrical conductor layer. One of more layers of the coating material layer may be bio-inert. One or more of the material layers may be hermitic. One or more of the layers of the coating material layer may be biocompatible. One or more of the layers may be a strengthening layer. One or more of the coating material layer may be functionalized to improve adhesion, proliferation, or differentiation of biological cells. One or more of the layers may be a low dissolution rate cell adhesion and proliferation layer. One or more of the layers may be an anti-inflammatory layer(s). One or more of the layer may be a bio-dissolvable layer(s). One example of a device having optional anti-inflammatory and dissolvable layers is seen in FIG. 5.

A portion of the electrode device may be outside of the physiological environment. The electrode device may be entirely within the physiological environment, for example, if the electrode device has wireless communication or wireless power transfer to outside of the physiological environment. One or more layers of the coating material may have a low dissolution layer or low etch rate in physiological environment. One or more layers of the coating material may have enhanced resistance to electrolysis in a physiological environment.

An electrode device structure to ensure that the electrode material does not electrically contact the electrically conducting low dissolution layer (e.g., titanium nitride) can have an electrical conducting layer laterally separated from the electrode material by a lateral etch of the electrically conducting low dissolution layer in the region of the opening of the outer electrical insulating low dissolution layer for the electrode material to contact the electrical conductor material layer.

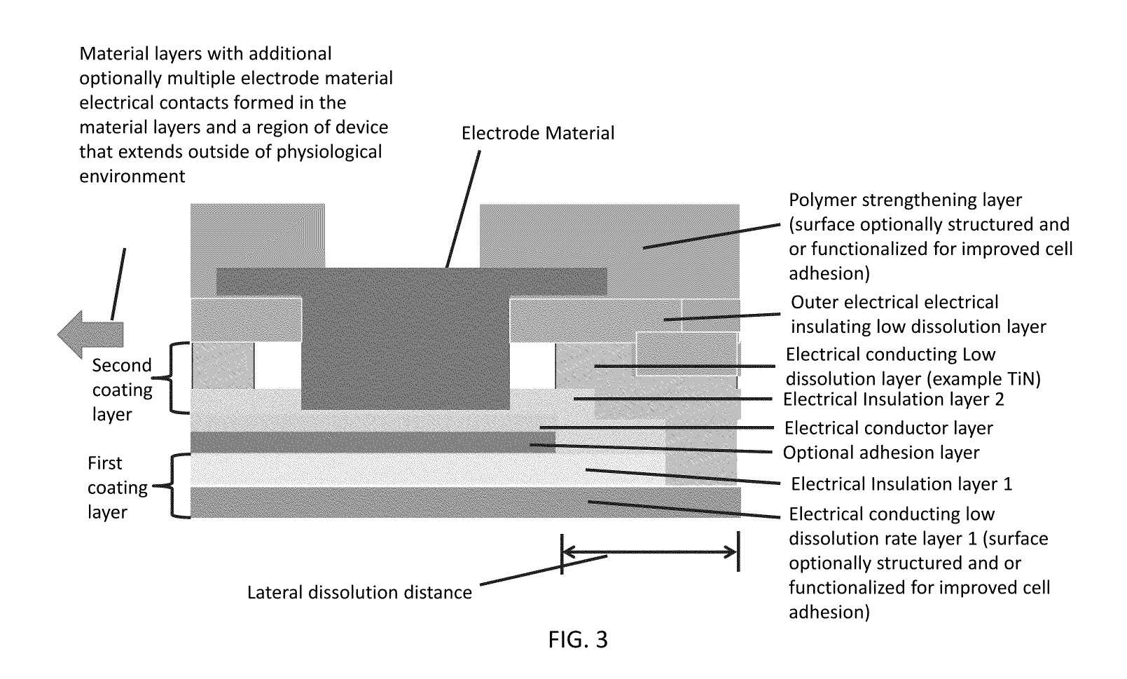

FIG. 3 shows an exemplary electrode device structure to ensure that an electrical conducting low dissolution layer (e.g., a layer of titanium nitride) does not electrically contact the electrode material. The electrode device structure is fabricated so that the outer electrical insulating low dissolution layer overlaps the electrical conducting low dissolution titanium nitride layer and insulates the electrical conducting low dissolution titanium nitride layer from the electrode material. The method to fabricate the electrode device structure with an outer electrical insulating layer that insulates the electrical conducting low dissolution layer from the electrode material is to use an additional photolithography and etch step to first define and etch the opening in the electrical conducting titanium nitride low dissolution layer and the electrical insulating layer to the electrical conductor layer, deposit the outer electrical insulating low dissolution layer and then use a second photolithography and define and etch step to etch the outer electrical insulating low dissolution layer to the surface of the electrical conductor layer so that the outer electrical insulating low dissolution layer overlaps the electrical conducting titanium nitride low dissolution layer.

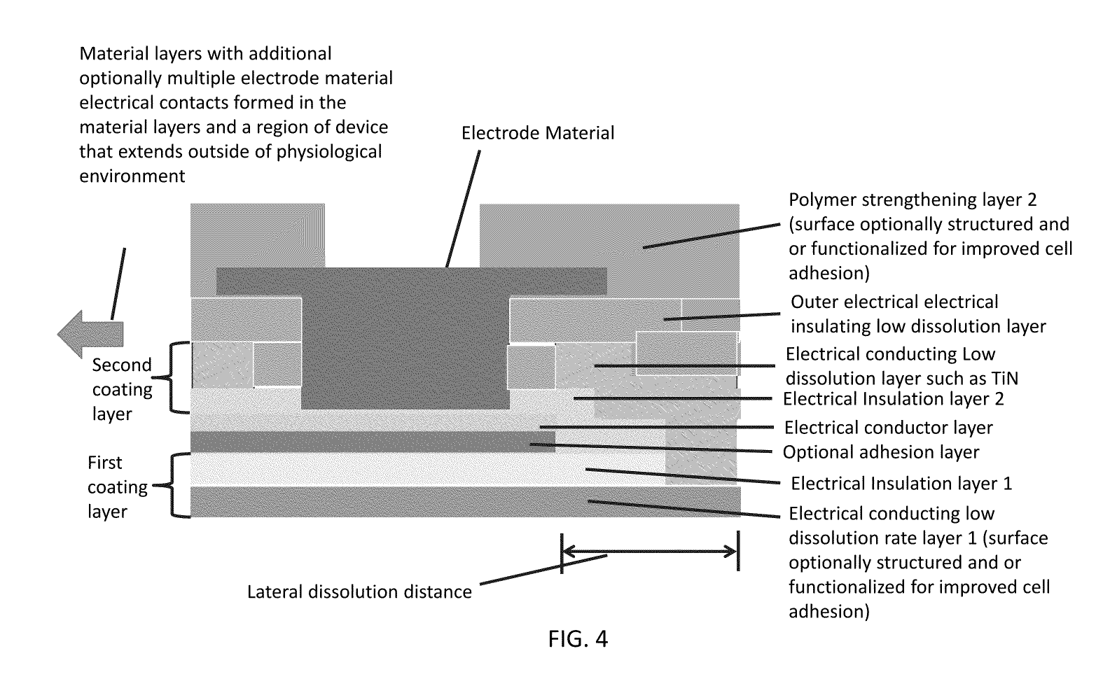

FIG. 4 shows another exemplary electrode device configures to isolate an electrical conducting low dissolution layer. In this case, an outer electrical insulating low dissolution layer overlaps the electrical conducting low dissolution titanium nitride layer and insulates the electrical conducting low dissolution titanium nitride layer from the electrode material. To fabricate the electrode device structure with an outer electrical insulating layer that insulates the electrical conducting low dissolution layer from the electrode material, a photolithography and etch step defines and etches the opening in the electrical conducting titanium nitride low dissolution layer and the electrical insulating layer to the electrical conductor layer. The outer electrical insulating low dissolution layer is deposited. Then additional photolithography and etching are performed so that the outer electrical insulating low dissolution layer overlaps the electrical conducting titanium nitride low dissolution layer.

The low dissolution rate layer(s) may partially or fully encapsulate the device. In some embodiments, the one or more low dissolution rate material layers encase the electrical conductor for the portion of the electrode device that is embedded in the physiological environment except for a region where the electrode material contacts an electrical conductor layer. The low dissolution rate layers may be displaced from the electrical conductor surface by a material layer or material layer(s). The material layer may include insulation layers, polymer layer, strengthening layer, or other material layer. A high level of adhesion of the material layer to the electrical conductor is desirable to prevent the physiological environment material laterally etching the electrical conductor at the material layer or at the interface of the electrical conductor and material layer.

In some embodiments, the one or more low dissolution rate material layers are in contact with the electrical conductor surfaces and encase the electrical conductor for the portion of the electrode device that is embedded in the physiological environment except for the region where the electrode material contacts the electrical conductor layer. The low dissolution rate material may have a high level of adhesion to surface of the electrical conductor.

In some embodiments, the one or more low dissolution rate material layers are in contact with electrical conductor surfaces and encase the electrical conductor for a portion of the electrode device that is embedded in the physiological environment while excluding (not encasing) a region where the electrode material contacts the electrical conductor layer. The low dissolution rate material may have a high level of adhesion to surface of the electrical conductor.

In some embodiments, the one or more low dissolution rate material layers are above and below the top and bottom surfaces of the electrical conductor except for the region where the electrode material contacts the electrical conductor layer but are displaced from the electrical conductor surface by a material layer. The material layer may include insulation layers, polymer layer, strengthening layer, or other material layer. A high level of adhesion of the material layer to the electrical conductor is desirable to prevent the physiological environment material laterally etching the electrical conductor at the material layer or at the interface of the electrical conductor and material layer.

In some embodiments, low dissolution rate cell adhesion and proliferation layers are on top and bottom surfaces.

One or more layers of the coating material may be an etch stop layer. One or more of the coating material may be a low damage etch layer in contact with an electrode material layer and can be etched without significant degradation of the surface of the electrode material.

The layers of the coating material may be deposited at a temperature less than 800° C. Alternately, the layers of the coating material may be deposited at a temperature less than 450° C. Alternately, the layers of the multilayer coating can be deposited at a temperature less than 300° C. Alternately, the layers of the coating material may be deposited at a temperature less than 200° C. Alternately, the layers of the coating material may be deposited at a temperature less than 100° C. Alternately, the layers of the coating material may be deposited at a temperature less than 50° C.

The low dissolution rate material may have a dissolution rate less than 3 nm/day at 96.4° C. while in an in vivo environment. The low dissolution rate material may have a dissolution rate less than 2 nm/day at 96.4° C. while in an in vivo environment. The low dissolution rate material may have a dissolution rate less than 1 nm/day at 96.4° C. while in an in vivo environment. Embodiments may have a low dissolution rate material may have a dissolution rate less than 0.1 nm/day, 0.01 nm/day, or 0.002 nm/day at 96.4° C. while in an in vivo environment.

The device may be less than 25 microns thick. The device may be less than 15 microns thick. The device may be less than 10 micron thick. The device may be less than 5 microns thick. The device may be less than 2 micron thick. The device may be less than 1 micron thick. The device may be less than 500 nm thick. The device may be less than 250 nm thick.

A circuit may be an electrical conductor. A circuit may be a microelectronic circuit. A circuit may be a multichip circuit. A circuit may be a hybrid circuit. A circuit may be a three-dimensional integrated circuit. A circuit may be a microelectromechanical circuit. A circuit may be a heterogeneous circuit. A circuit may include an antenna. A circuit may include apparatus for electrical energy storage such as an ultracapacitor or a battery. A circuit may include a sensor or stimulator.

An embodiment, an optional low damage etch layer is in contact with an electrode material surface. The low damage etch layer can be etched using etch approaches that have low levels of damage to the surface of the surface of an electrode material. In an embodiment, a layer can also be an optional etch stop layer. In an embodiment, a layer can be an optional insulation layer. In an embodiment, a layer can be a low dissolution rate layer. The low dissolution rate layer can be a bio-inert layer. In an embodiment, a layer can be an optional polymer biocompatible layer. In an embodiment, a layer can be an anti-inflammatory layer. In an embodiment, a layer can be a dissolvable layer.

A substrate may comprise silicon, gallium nitride, silicon carbide, polymer, polyimide, diamond, or combinations thereof. The silicon substrate may be less than about 4 nm thick. The silicon substrate may be less than about 20 nm thick. The silicon substrate may be less than about 40 nm thick. The silicon substrate may be less than about 100 nm thick. The silicon substrate may be less than about 200 nm thick. The silicon substrate may be less than about 500 nm thick. The silicon substrate may be less than about 1000 nm thick. The silicon substrate may be less than about 2000 nm thick. The silicon substrate may be less than about 5 micron thick. The silicon substrate may be less than about 10 micron thick. The silicon substrate may be less than about 20 micron thick. The silicon substrate may be less than about 50 micron thick. The substrate may be flexible. The substrate may comprise transistor devices. The transistor devices may comprise metal interconnected field effect transistor (FET) arranged in circuit configuration. The circuit configuration may perform the function of sequential addressing and reading the electrical signal from electrodes.

The substrate may be a multilayer substrate. A multilayer substrate may include a stacked layer structure. A stacked layer structure may include diamond coated on the first side of silicon material. A stacked layer structure may include diamond coated on the second side of a silicon layer. A stacked layer structure may include diamond coated on a first and second side of a silicon material. A stacked layer structure may include a polymer coated on the first side of a silicon material. A stacked layer structure may include a polymer coated on the second side of a silicon layer. A stacked layer structure may include a polymer coated on the first and second side of a silicon layer.

An electrical conductor may comprise gold, platinum, platinum/iridium alloy, stainless steel, aluminum, iridium, titanium, metal, conductive semiconductor, conductive silicon, doped silicon, doped polysilicon, doped diamond, carbon nanotubes, single-wall carbon nanotube, multi-wall carbon nanotube, binder-free carbon nanotube interconnected layer, carbon nanotube interconnected layer with binder, non-aligned carbon nanotube, aligned carbon nanotubes, deposited carbon nanotubes, graphene, diamond like carbon, carbon nanostructured material, conductive colloid, conductive ink, conductive polymer, and combinations thereof. The electrical conductor material may be a polycrystalline, nanocrystalline, amorphous, highly-oriented, two-dimensional, composite, or single-crystal material layer. The implanted electrode may have multiple levels of electrical conductor with insulation between each level of electrical conductor. The approach for depositing the electrical conductor material should be compatible with the material in the multilayer coating. For example, if an aluminum release layer is used, approach that deposit electrical conductor material at a temperature of about 475° C. may be used. The linear coefficient of the electrical conductor should also be compatible with the linear coefficient of expansion of the material in the multilayer coating. For example, a deposit carbon nanotube electrical conductor may be advantageous linear coefficient of thermal expansion that can be compatible with the linear coefficient of expansion of many polymer layers.

A release layer may include but not be limited to aluminum, alloy, aluminum alloy, aluminum-copper alloy, aluminum-silicon alloy, copper, copper alloy, nickel, nickel alloy, transition metal, transition metal alloy, silicon, polysilicon, silicon oxide, polymer, polymer resist, and combinations thereof. The release layer may be selected to be compatible with a selected processing temperature. For example, an insulation layer may be deposited using an atomic layer deposition which typically has process temperatures that range from room temperature to about 450° C. An aluminum release layer can be compatible with an atomic layer deposition temperature. Some atomic layer deposition tools have the capability for 1000° C. deposition temperature. A polysilicon release layer would be compatible with a 1000° C. deposition temperature. A copper release layer would be compatible with about 900° C. processing temperature. There may be a dielectric layer such as silicon oxide or silicon nitride on the substrate between the release layer and the substrate to minimize the reaction of the release layer material with the substrate.

A strengthening layer may include a polymer layer, a polysilicon layer, a semiconductor layer, and a dielectric layer. The strengthening layer may provide mechanical support to the material layers within an electrode device. The strengthening layer may allow the electrode device to be flexible without cracking of the material layers and/or provide sufficient strength such that the device can survive normal handling.

An electrode preferably comprises an electrode material, which may comprise iridium material, iridium alloy material, iridium/platinum alloy material, iridium oxide material, activated iridium oxide material, tungsten material, platinum material, platinum black material, titanium nitride material, silver/silver chloride material, conductive diamond material, P-type doped diamond material, titanium material, titanium nitride material, carbon nanostructures material, carbon nanotube material, graphene material, graphene nanoplatelets material, and combinations thereof. A electrode material may comprise but not limited to a layer, nanotube, nanostructures, wire, micro-wire. The approach to form the electrode material may include but not be limited to electrodeposition, electroplating, sputtering, e-beam evaporation, ion beam deposition, etching, and sharpening. The electrode material may be nanowire electrode material.

An activated iridium oxide layer (suitable as an electrode material) may be formed by electrochemical conversion of a portion of iridium metal to an iridium oxide layer. The impedance of the iridium electrode material can be reduced by a factor of 10 by forming activated iridium oxide layer. An iridium oxide layer may be formed by thermal decomposition of iridium salts. The electrode material may be use for stimulating physiological response. The electrode material may be used for sensing. The implantable electrode may have one of more electrode material sites. The electrode material site is a region of the implantable electrode where the electrode material can interact with physiological material.

In some embodiments of a device, an deposited electrode material is not required. The electrical conductor can include electrode material.

In some devices such as a strain sensor, electrode material is not required. The electrical conductor or the substrate can be a strain sensor.

In some embodiments, the electrode material may also be an electrical conductor (such as the above-referenced conductor). In some embodiments, the electrical conductor may be an electrical interconnect. In some embodiments, the electrical conductor may be a substrate. In some embodiments, the electrical conductor may be flexible. Some embodiments may include a flexible interconnect to facilitate floating implantable electrodes.

A lateral dissolution distance can be selected to be compatible with device lifetime. A dissolution rate is typically specified in nm/day at a temperature in a specified environment. The lateral dissolution distance is typically much larger than a vertical dissolution distance. Thus, for many embodiments, the lateral dissolution distance can be selected for a selected device lifetime. Insulating material such as Al₂O₃ can have a dissolution rate less than 5 nm/day in a physiological environment. Thus, a device lifetime of 1000 days would require a lateral dissolution distance of 5000 nm or approximately 5 microns. In some embodiments, the lateral dissolution distance can be less than 100 nm. In some embodiments, the lateral dissolution distance can be less than 500 nm. In some embodiments, the lateral dissolution distance can be less than 1000 nm. In some embodiments, the lateral dissolution distance can be less than 5000 nm. In some embodiments, the lateral dissolution distance can be less than 50 microns. In some embodiments, the lateral dissolution distance can be less than 1000 microns.

The vertical dissolution distance may be the thickness of a low dissolution layer. The vertical dissolution distance may be thickness of a low dissolution layer and additional layers such as an insulation layer or a polymer layer or combination of layers.

The adhesion between two material layers can be optimized to prevent the lateral dissolution of a material layer at the interface of two material layers. In some embodiments, an adhesion promoter material layer can be applied to a first material surface to facilitate the adhesion of a second material layer to the first material surface. In some embodiments an adhesion promoter can be applied to an inorganic material layer surface to increase the adhesion of a polymer layer to the inorganic material layer surface and minimize the lateral dissolution of the material layers at the interface.

A low damage etch layer may include but not be limited to silicon oxide, silicon nitride, polysilicon, aluminum oxide and combinations thereof. A characteristic of the low damage etch layer is that the low damage etch layer can be etched without significantly damaging the surface of the electrode material.

An etch stop layer may include but not be limited to a compound oxide layer, a compound nitride layer, metal oxide layer, a silicon oxide layer, a silicon nitride layer, a boron nitride layer, an aluminum nitride layer, an aluminum oxynitride layer, and combination thereof. The etch stop layer has an etch rate that is less than the etch rate of the optional electrical insulation layer in the etchant used to etch the electrical insulation layer. Alternately, the etch stop layer has an etch rate that is less than the etch rate of the low dissolution rate layer if the low dissolution rate layer is also an insulation layer and an electrical insulation layer is not included in the multilayer coating.

An electrical insulation layer may include but not be limited to a compound oxide layer, a compound nitride layer, metal oxide layer, a silicon oxide layer, a silicon nitride layer, a boron nitride layer, a zirconium oxide layer, an aluminum nitride layer, an aluminum oxynitride layer, and combination of thereof. The electrical insulation layer may include one or more layers of electrical insulation material layers. The electrical insulation layer may provide electrical insulation of an electrical conductor from the material in the adjacent environment including electrical insulation physiological environment.

A low dissolution rate layer may include (but is not limited to) a gallium containing layer, boron containing layer, nitride containing layer, oxide containing layer, zirconium containing layer, aluminum containing layer, titanium containing layer, gallium nitride, boron nitride, zirconium oxide, diamond, aluminum oxide, titanium nitride, titanium carbide, titanium dioxide, and combinations thereof. The low dissolution rate layer may comprise one or more low dissolution rate layers. The method to deposit the low dissolution rate layer may include but not be limited to chemical vapor deposition (CVD), Metal Organic Chemical Vapor Deposition, Microwave Plasma Chemical Vapor Deposition, Hot Filament Chemical Vapor Deposition, atomic layer deposition (ALD), and atomic layer epitaxy (ALE). The etch dissolution rate layer may have a low density pins or may be pinhole free. The low dissolution rate layer may be a conformal layer and have the ability to coat three-dimensional surface. The low dissolution rate layers may be deposited at a temperature less than 800 C. The low dissolution rate layers may be deposited at a temperature less than 450 C. The low dissolution rate layers may be deposited at a temperature less than 450 C. The low dissolution rate layers may be deposited at a temperature less than 450 C. The low dissolution rate layers may be deposited at a temperature less than 300 C. The low dissolution rate layers may be deposited at a temperature less than 200 C. The low dissolution rate layers may be deposited at a temperature less than 100 C. The low dissolution rate layers may be deposited at a temperature less than 50 C. The low dissolution rate material may have a dissolution rate less than 3 nm/day at 96.4° C. in an in vivo environment. The low dissolution rate material may have a dissolution rate less than 2 nm/day at 96.4° C. in an in vivo environment. The low dissolution rate material may have a dissolution rate less than 1 nm/day at 96.4° C. in an in vivo environment.

The low dissolution layer cell may also have surface characteristics that optimize cell adhesion, cell proliferation, and cell differentiation. A layer providing such characteristics may be known as a low dissolution cell adhesion and proliferation layer. For example, a gallium nitride layer is a low dissolution rate material and the surface properties may be modified for cell adhesion, cell proliferation, and cell differentiation. The cell adhesion is often improved if the surface of the low dissolution layer has a non-planar topography, such as roughness. The biocompatible polymer layer can be structured photolithography patterning followed by oxygen plasma etching and then an atomic layer deposited GaN layer deposited on the surface of the biocompatible layer. The biocompatible polymer layer surfaces can also be modified for cell adhesion, cell proliferation and cell differentiation.

A polymer layer may include but not be limited to a parylene layer, parylene-C layer, benzocyclobutene (BCB) layer, PDMS layer, polyimide layer, liquid crystal polymer layer, poly(3,4-ethylenedioxythiophene (PEDOT), polylysine, polypyrrole, hydrogel, and combinations thereof (see P. Anikeeva, “Biocompatible Materials for Optoelectronic Neural Probes: Challenges and Opportunities” The Bridge Vol. 43, No. 4, Winter 2013, pp. 39-48). The polymer layer may provide mechanical strength to a flexible electrode device. For example, a layer of the polymer on attached to electrical conductor and low dissolution rate material layer(s) will allow the electrode device to be flexible without breaking when bent. A flexible electrode is advantageous for implantable electrode because it will cause less strain on the tissue adjacent to the electrode device. The polymer layer may be a biocompatible polymer layer. A biocompatible polymer layer is desirable for implantable electrodes. The biocompatible polymer layer may have low modulus characteristics. A low modulus polymer is desirable to because it will cause less strain on the tissue adjacent to the electrode device.

It can be desirable to match the mechanical properties of implanted devices/electrodes and the tissue in which they are implanted (for example, brain tissue) in order to avoid strain at their interface and disruptions in transmission to and from the tissue. The shear modulus of the brain is 200 to 1500 Pa. See Grill W M. “Signal Considerations for Chronically Implanted Electrodes for Brain Interfacing.” in Indwelling Neural Implants: Strategies for Contending with the In Vivo Environment. Boca Raton (Fla.): CRC Press; 2008. Chapter 2.

The polymer layer may be selected to be a low swelling polymer in the selected environment of the device. The polymer layer may be selected to be a low water absorption in the selected environment of the device. A low swelling polymer can be desirable to prevent cracking of material layers adjacent to the polymer layer. The polymer layer may be protected by a material layer from exposure to the environment. A low dissolution rate layer that is deposited on the surface of the polymer can minimize the interaction of the polymer with the environment. The polymer layer may be selected to have a thermal coefficient of expansion value that is compatible with the material layers in the device. For example, a polyethylene naphthalate (PEN) polymer may be compatible with the thermal expansion coefficient of silicon.

The polymer layer may be selected to be compatible with the processing temperature. For the case that a low dissolution rate material is deposited after then polymer layer is formed, it is desirable that the polymer layer be compatible with the deposition temperature of the low dissolution rate material.

The electrode device may be thin. A thin electrode device is desirable because it will cause less strain on the tissue adjacent to the electrode device. The electrode device may be less than 25 microns thick. The electrode device may be less than 15 microns thick. The electrode device may be less than 10 micron thick. The electrode device may be less than 5 microns thick. The electrode device may be less than 2 micron thick. The electrode device may be less than 1 micron thick. The electrode device may be less than 500 nm thick. The electrode device may be less than 250 nm thick. See Greco, F. et al., “Patterned Free-Standing Conductive Nanofilms for Ultraconformable Circuits and Smart Interfaces,” ACS Applied Materials & Interfaces 2013 5 (19), 9461-9469 and Pensabene, V. et al., “Flexible polymeric ultrathin film for mesenchymal stem cell differentiation,” Acta Biomaterialia, Volume 7, Issue 7, July 2011, Pages 2883-2891.

The polymer film may be a nanofilm. The polymer layer may comprise nanoparticles inside of the polymer to enhance the adhesion and proliferation of biological cells. Ventrelli et al., “Influence of nanoparticle-embedded polymeric surfaces on cellular adhesion, proliferation, and differentiation.” J Biomed Mater Res Part A 2014: 102A: 2652-2661. The polymer layer may comprise carbon nanotubes, graphene, or graphene nanoplatelets inside of the polymer film. The carbon nanotubes, graphene, or graphene nanoplatelets may enhance the mechanical strength.

An anti-inflammatory coating may include in the multilayer coating. The anti-inflammatory layer may reduce glial scar formation in the vicinity of the implanted electrode. The anti-inflammatory coating may include but not be limited to a nitrocellulose-based dexamethasone coating, peptide coating, hydrogel, functionalized hydrogel, and combinations thereof.

A bio-dissolvable layer may include but not be limited to PEG, sugar, silk fibroin or cellulose or combinations thereof. An advantage of the bio-dissolvable layer is that the material can temporarily stiffen the probe to facilitate implantation. See P. Anikeeva, previously cited. The bio-dissolvable material may be a microneedle. See Xiang, Z. et al., “Ultra-thin flexible polyimide neural probe embedded in a dissolvable maltose-coated microneedle,” J. Micromech. Microeng. 24 (2014) 065015.

In an embodiment, an etch stop layer may comprise a low damage etch layer.

In an embodiment, an electrical insulation layer may comprise an etch stop layer.

In an embodiment, a low dissolution rate layer may comprise an electrical insulation layer. In an embodiment, the low dissolution rate layer may comprise a cell adhesion, cell proliferation, and cell differentiation layer.

The device may include a light source.

Contemplated herein are methods of using the above-described devices, including for example implanting them in an animal (optionally a mammal, for example a human), recording signals from such a device, causing such devices to send stimulations, etc.

### Methods of Making

A method to fabricate the device may comprise planar processing approaches. An advantage of planar processing approaches is that multiple devices can be fabricated in simultaneously using microelectronic or microelectromechanical (MEMS) processing techniques. The method of fabricating may include wafer level processing. Wafer level processing enables a large number of devices to be fabricated simultaneously, reducing the cost of making each item.

In some embodiments, the device may be coated with one or more low dissolution rate layer(s) at the die configuration, three-dimensional integrated circuit configuration, or three dimensional package configuration rather than the wafer level. One method to coat a die configuration device, a three-dimensional configuration device, or a three-dimensional package configuration with a low dissolution rate layer(s) is to utilize a fluidized bed atomic layer deposition or rotary atomic layer deposition to coat surfaces of the die configuration device or three-dimensional integrated circuit configuration device with a low dissolution rate material. An advantage of the fluidized bed atomic layer deposition or rotary atomic layer deposition approach is that it tends to result in surfaces of the device being coated with a pin-hole free, highly conformal, atomic layer deposited film, with the ability to apply different material coats in succession. The three-dimensional integrated circuit configuration device may comprise stacked electrically active device layers with each electrically active device layer optionally having through-silicon-vias joined by bonded or soldered electrical interconnects optionally having interposer electrical interconnects utilizing through-silicon-via between the stacked electrical active device layers. The three-dimensional package configuration may include wire bond or solder electrical interconnects between electrical active device die.

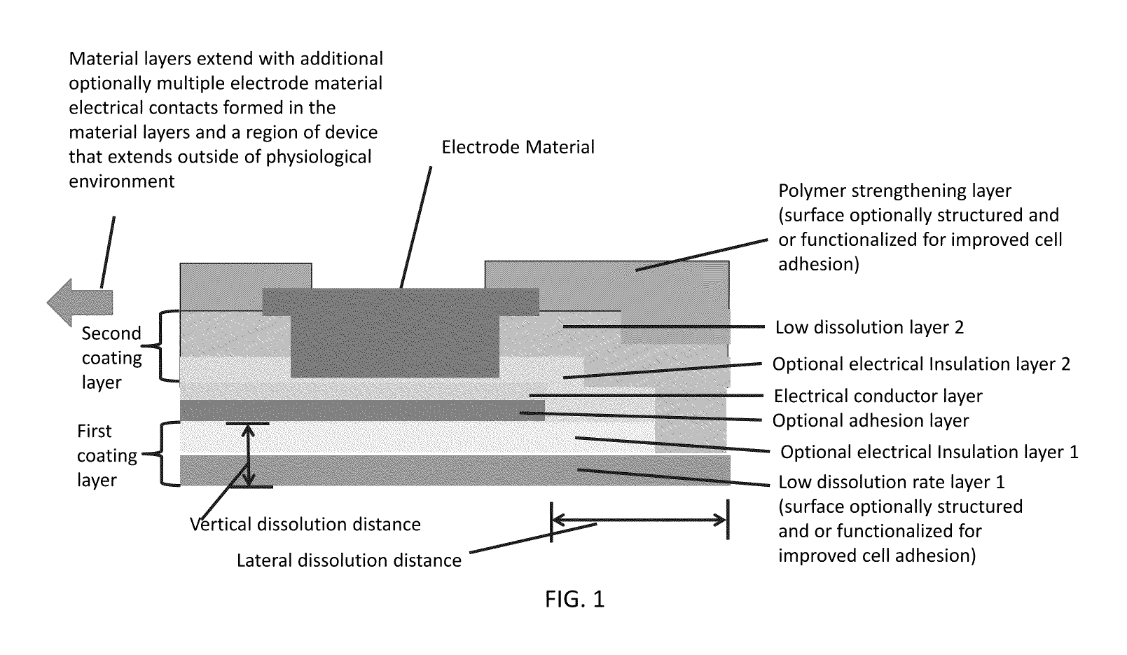

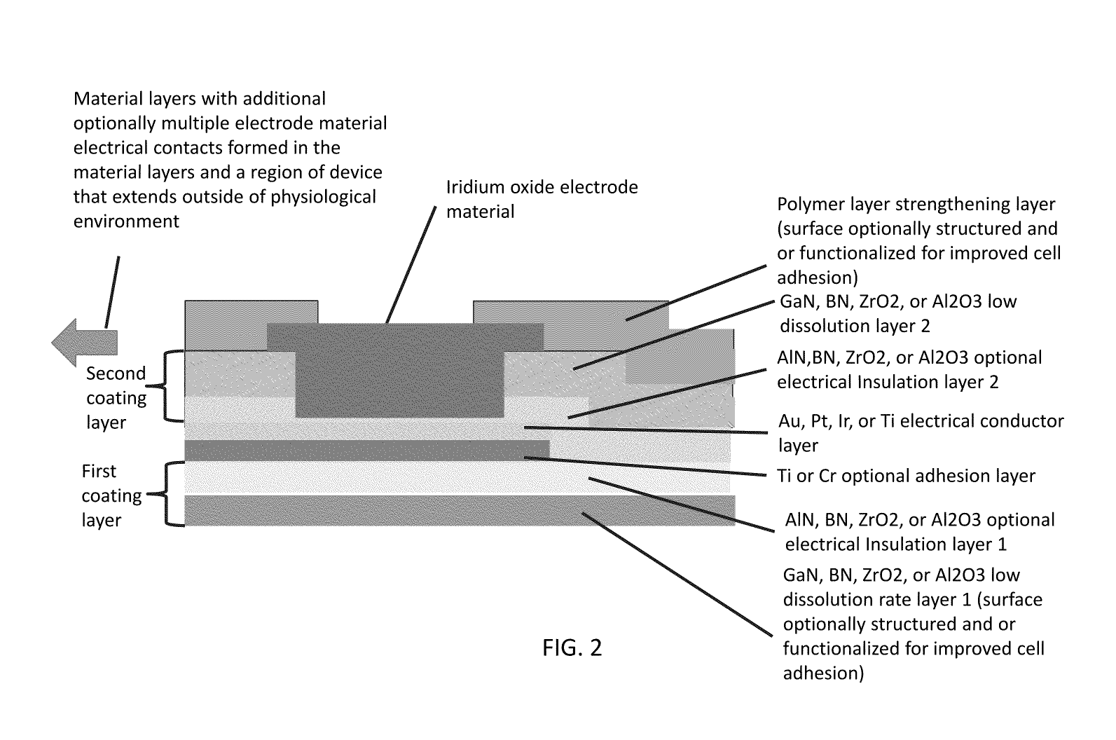

First prophetic exemplary process (at least partially applicable to FIGS. 1 and 2): outline of steps to fabricate flexible implantable electrode with low dissolution rate layers on both sides of metal conductor and polymer layer on top surface.

1. Deposit a release layer (for example, an aluminum layer or polymer) on a silicon substrate.

2. Deposit low dissolution rate layer 1.

3. Optionally deposit insulation layer 1.

4. Optionally deposit strengthening material layer.

5. Deposit titanium or chrome adhesion metal (this can improve the adhesion of a second deposited metal).

6. Deposit electrical conductor (for example, gold, platinum, iridium, and/or titanium).

7. Perform photolithography step to define and etch the electrical conductor and the adhesion metal.

8. Optionally deposit insulation layer 2.

9. Optionally perform lithography step to define and etch insulation layer 1 and insulation 2 to low dissolution layer 1

10. Deposit low dissolution rate layer 2.

11. Deposit polymer layer 2

12. Optionally deposit anti-inflammatory layer.

13. Perform photolithography step and etch step to pattern the anti-inflammatory layer, polymer layer, the optional strengthening layer, and the low dissolution layer 1 to the release layer.

14. Perform photolithography step to form a contact window to the electrical conductor.

15. Optional form electrode material by method including but not limited to electrodeposit, sputtering, and photolithography and etching or liftoff.

16. Optionally functionalize the exposed surface of the polymer layer, the low dissolution layer, and the optionally anti-inflammatory layer for improved cell adhesion and cell proliferation.

17. Optionally deposit anti-inflammatory layer if not deposited in an earlier step.

18. Optional photolithography step to define and etch a contact window in the anti-inflammatory layer to the electrode material.

19. Optionally adhere to support material (include but not limited to UV releasable tape or heat releasable tape or electrostatic holder) to facilitate handling.

20. Release the implantable electrode device from the silicon substrate by a method including but not limited to etching the release layer or by electrochemically dissolving the release layer.

21. Optionally deposit anti-inflammatory layer.

22. Optionally functionalize surfaces for cell adhesion, replication and differentiation.

23. Optionally deposit bio-dissolvable material, adhere bio-dissolvable material, or use bio-dissolvable adhesion material to adhere a stiffener to temporarily stiffen implantable electrode device to facilitate implant of the electrode device. The bio-dissolvable material can be deposited on one surface, more than one surface, or all surfaces.

24. Optionally dice into individual sensors

25. Optionally release from support material. The release methods may include but not be limited ultraviolet illumination for ultraviolet releasable tape, heating for heat releasable tape, dissolving of a polymer layer, or release of the electrical energy for an electrostatic chuck.

Second prophetic exemplary process (at least partially applicable to FIGS. 3 and 4): outline of steps to fabricate flexible implantable electrode with electrically conductive low dissolution rate layers on both sides of metal conductor and polymer layers on top and bottom surfaces.

1. Deposit a release layer (for example, an aluminum layer or polymer) on a silicon substrate.

2. Deposit electrically conductive low dissolution rate layer 1.

3. Deposit insulation layer 1.

4. Optionally deposit strengthening layer 1.

5. Deposit titanium or chrome adhesion metal (this can improve the adhesion of a second deposited metal).

6. Deposit electrical conductor (for example gold, platinum, iridium, and/or titanium).

7. Perform photolithography step to define and etch the electrical conductor and the adhesion metal.

8. Deposit insulation layer 2.

9. Optionally perform lithography step to define and etch insulation layer 1 and insulation layer 2 to low dissolution layer 1.

10. Deposit electrically conductive low dissolution rate layer 2.

11. Deposit insulation layer 3.

12. Deposit polymer strengthening layer 2.

13. Optionally deposit anti-inflammatory layer.

14. Perform photolithography step and etch step to pattern the anti-inflammatory layer, polymer layer, the optional strengthening layer, and the low dissolution layer 1 to the release layer

15. Perform photolithography step to form a contact window to the electrical conductor.

16. Optional form electrode material by method including but not limited to electrodeposit, sputtering, and photolithography and etching or liftoff.

17. Optionally functionalize the exposed surface of the polymer layer, the low dissolution layer, and the optionally anti-inflammatory layer for improved cell adhesion and cell proliferation.

18. Optionally deposit anti-inflammatory layer if not deposited in an earlier step.

19. Optional photolithography step to define and etch a contact window (via window) in the anti-inflammatory layer to the electrode material.

20. Optionally adhere to support material (include but not limited to UV releasable tape or heat releasable tape or electrostatic holder) to facilitate handling.

21. Release the implantable electrode device from the silicon substrate by a method including but not limited to etching the release layer or by electrochemically dissolving the release layer.

22. Optionally deposit anti-inflammatory layer.

23. Optionally functionalize surfaces for cell adhesion, replication and differentiation.

24. Optionally deposit bio-dissolvable material, adhere bio-dissolvable material, or use bio-dissolvable adhesion material to adhere a stiffener to temporarily stiffen implantable electrode device to facilitate implant of the electrode device. The bio-dissolvable material can be deposited on one surface, more than one surface, or all surfaces.

25. Optionally dice into individual sensors.

26. Optionally release from support material. The release methods may include but not be limited ultraviolet illumination for ultraviolet releasable tape, heating for heat releasable tape, dissolving of a polymer layer, or release of the electrical energy for an electrostatic chuck.

Third prophetic exemplary process: outline of steps to fabricate a flexible implantable electrode with biocompatible layer on both sides of electrical conductor and a low dissolution rate cell adhesion and proliferation layer on surface of biocompatible material layers.

1. Deposit a release layer (for example, an aluminum layer or polymer) on a silicon substrate.

2. Deposit polymer strengthening layer 1.

3. Deposit low dissolution layer 1 at a temperature that is compatible with biocompatible polymer layer 1. An atomic layer deposition approach can deposit at temperature compatible with biocompatible layer 1.

4. Optionally deposit insulation layer 1

5. Optionally deposit strengthening material layer.

6. Deposit titanium or chrome adhesion metal (this can improve the adhesion of a second deposited metal).

7. Deposit electrical conductor (for example, gold, platinum, iridium, and/or titanium).

8. Perform photolithography step to define and the electrical conductor and the adhesion metal.

9. Optionally deposit insulation layer 2 at a temperature compatible with polymer layer 1

10. Optionally perform lithography step to define and etch insulation layer 1 and insulation 2 to low dissolution layer 1

11. Deposit low dissolution rate layer 2 at a temperature that is compatible with polymer layer 1.

12. Deposit polymer strengthening layer 2.

13. Optionally deposit anti-inflammatory layer.

14. Perform photolithography step and etch step to pattern the anti-inflammatory layer, polymer layer, the optional strengthening layer, and the low dissolution layer 1 to the release layer.

15. Perform photolithography step to form a contact window to the electrical conductor.

16. Optional form electrode material by method including but not limited to electrodeposit, sputtering, and photolithography and etching or liftoff.

17. Optionally functionalize the exposed surface of the polymer layer, the low dissolution layer, and the optionally anti-inflammatory layer for improved cell adhesion and cell proliferation.

18. Optionally deposit anti-inflammatory layer if not deposited in an earlier step.

19. Optional photolithography step to define and etch a contact window in the anti-inflammatory layer to the electrode material.

20. Optionally adhere to support material (include but not limited to UV releasable tape or heat releasable tape or electrostatic holder) to facilitate handling.

21. Release the implantable electrode device from the silicon substrate by a method including but not limited to etching the release layer or by electrochemically dissolving the release layer.

22. Optionally deposit anti-inflammatory layer.

23. Optionally functionalize surfaces for cell adhesion, replication and differentiation.

24. Optionally deposit bio-dissolvable material, adhere bio-dissolvable material, or use bio-dissolvable adhesion material to adhere a stiffener to temporarily stiffen implantable electrode device to facilitate implant of the electrode device. The bio-dissolvable material can be deposited on one surface, more than one surface, or all surfaces.

25. Optionally dice into individual sensors.

26. Optionally release from support material. The release methods may include but not be limited ultraviolet illumination for ultraviolet releasable tape, heating for heat releasable tape, dissolving of a polymer layer, or release of the electrical energy for an electrostatic chuck.

Fourth prophetic exemplary process: outline of steps to fabricate flexible implantable electrode with low dissolution rate layers on both sides of metal conductor and low dissolution rate layer on surfaces.

1. Deposit a release layer (for example, an aluminum layer or polymer) on a silicon substrate.

2. Structure the release layer by patterning and etch a portion of the release layer.

3. Deposit low dissolution rate layer 1.

4. Optionally deposit insulation layer 1.

5. Optionally deposit strengthening material layer.

6. Deposit titanium or chrome adhesion metal (this can improve the adhesion of a second deposited metal).

7. Deposit electrical conductor (for example, gold, platinum, iridium, and/or titanium).

8. Perform photolithography step to define and the electrical conductor and the adhesion metal.

9. Optionally deposit insulation layer 2

10. Optionally perform lithography step to define and etch insulation layer 1 and insulation 2 to low dissolution layer 1.

11. Deposit low dissolution rate layer 2.

12. Deposit polymer strengthening layer 2

13. Optionally pattern the polymer layer by performing a photolithography step and etching a portion of the polymer layer.

14. Deposit a low dissolution rate layer 3.

15. Perform photolithography step and etch step to pattern the anti-inflammatory layer, polymer layer, the optional strengthening layer, and the low dissolution layer 1 to the release layer. Laterally undercut the release layer by etching the release layer laterally.

16. Deposit low dissolution rate layer 4 (preferably deposited using atomic layer deposition).

17. Anisotropic plasma etch low dissolution rate layer 4 so that dissolution rate layer 4 remains on the vertical sidewall and at least a portion of the underside of electrode device.

18. Optionally deposit anti-inflammatory layer.

19. Perform photolithography step and etch step to pattern the anti-inflammatory layer, polymer layer, the optional strengthening layer, and the low dissolution layer 1 to the release layer.

20. Perform photolithography step to form a contact window to the electrical conductor.

21. Optional form electrode material by method including but not limited to electrodeposit, sputtering, and photolithography and etching or liftoff.

22. Optionally functionalize the exposed surface of the polymer layer, the low dissolution layer, and the optionally anti-inflammatory layer for improved cell adhesion and cell proliferation.

23. Optionally deposit anti-inflammatory layer if not deposited in an earlier step.

24. Optional photolithography step to define and etch a contact window in the anti-inflammatory layer to the electrode material.

25. Optionally adhere to support material (include but not limited to UV releasable tape or heat releasable tape or electrostatic holder) to facilitate handling

26. Release the implantable electrode device from the silicon substrate by a method including but not limited to etching the release layer or by electrochemically dissolving the release layer.

27. Optionally deposit anti-inflammatory layer.

28. Optionally functionalize surfaces for cell adhesion, replication and differentiation.

29. Optionally deposit bio-dissolvable material, adhere bio-dissolvable material, or use bio-dissolvable adhesion material to adhere a stiffener to temporarily stiffen implantable electrode device to facilitate implant of the electrode device. The bio-dissolvable material can be deposited on one surface, more than one surface, or all surfaces.

30. Optionally dice into individual sensors.

31. Optionally release from support material. The release methods may include but not be limited ultraviolet illumination for ultraviolet releasable tape, heating for heat releasable tape, dissolving of a polymer layer, or release of the electrical energy for an electrostatic chuck.

Fifth prophetic exemplary process: outline of steps to fabricate flexible implantable electrode with low dissolution rate layers on both sides of metal conductor and low dissolution rate layer on surfaces.

1. Deposit a release layer (for example, an aluminum layer or polymer) on a silicon substrate.

2. Deposit low dissolution rate layer 1.

3. Optionally deposit insulation layer 1.

4. Optionally deposit strengthening material layer 1

5. Deposit titanium or chrome adhesion metal (this can improve the adhesion of a second deposited metal).

6. Deposit electrical conductor (for example, gold, platinum, iridium, and/or titanium).

7. Perform photolithography step to define and etch the electrical conductor and the adhesion metal.

8. Optionally deposit insulation layer 2.

9. Optionally perform lithography step to define and etch insulation layer 1 and insulation 2 to low dissolution layer 1.

10. Optionally deposit low dissolution rate layer 2.

11. Deposit polymer strengthening layer 2.

12. Perform photolithography step and etch step to patter polymer layer, the optional strengthening layer, and the low dissolution layer 1 to the release layer.

13. Peel device structure release layer or release the implantable electrode device from the silicon substrate by etching the release layer or by electrochemical dissolving the release layer.

14. Deposit low dissolution layer 2 using atomic layer deposition tool at a deposition temperature compatible with polymer layer 2 to achieve three-dimensional coating of the device structure.

15. Optionally deposit anti-inflammatory layer if not deposited in an earlier step.

16. Attach device structure an ultraviolet releasable tape or attach device structure to a heat releasable tape.

17. Perform photolithography step to form a contact window to the electrical conductor

18. Deposit electrode material

19. Perform step to pattern the electrode material or alternately use lift-off process for electrode material

20. Optionally deposit anti-inflammatory layer if not deposited in an earlier step.

21. Optional photolithography step to define and etch a contact window in the anti-inflammatory layer to the electrode material.

22. Release the device structure from UV releasable tape or heat releasable tape.

23. Optionally functionalize the exposed surface of the polymer layer, the low dissolution layer 1, and the optionally anti-inflammatory layer for improved cell adhesion and cell proliferation.

24. Optionally deposit dissolvable bio-dissolvable layer. The bio-dissolvable layer can temporarily strengthen the flexible implant electrode.

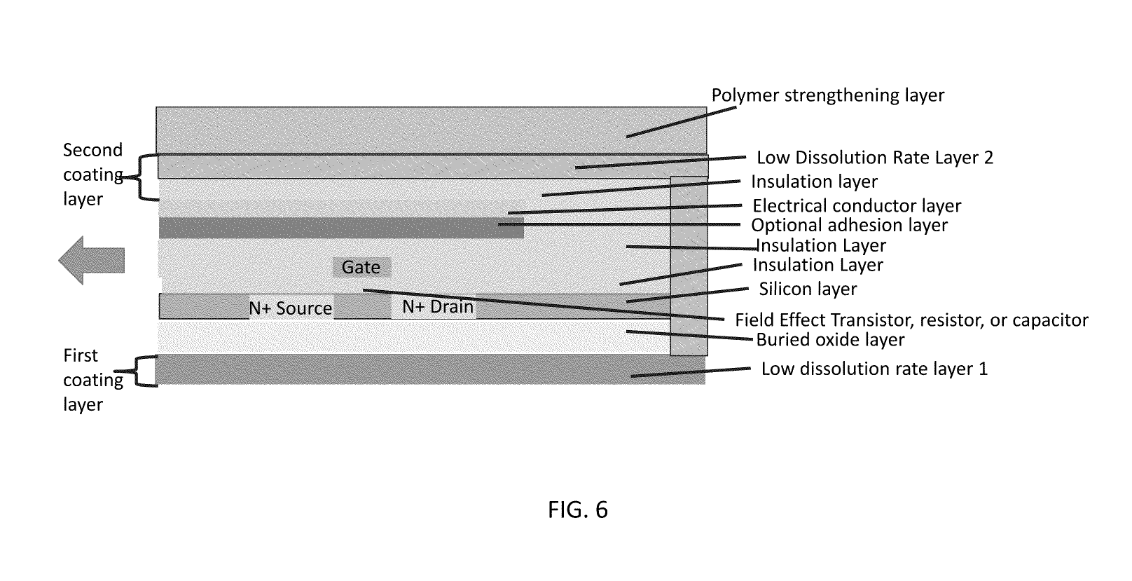

Sixth prophetic exemplary process (at least partially applicable to FIG. 6): outline of steps for forming a silicon-on-insulator (SOI) device having low dissolution rate protective layers optionally using electrostatic holder (optionally flexible).

1. Fabricate a silicon-on-insulator device or circuit optionally having a handle substrate, a buried oxide layer, a silicon device layer, N+ or P+ doped junction, a gate insulator, a gate electrode, electrode contacts to N+ or P+ doped junctions, an insulating layer over the gate of the device, an optical waveguide, a photodetector, a piezoelectric material layer, a magnetoresistive layer, a ferrite layer, a magnetic layer, a graphene layer, graphene nanoplatelets layer, a carbon nanotube layer, a high-K layer, a ferroelectric layer, or a ferromagnetic layer.

2. Optionally pattern and etch insulation layers, silicon layer and buried oxide layer to handle substrate.

3. Deposit Low Dissolution Rate Layer 2.

4. Deposit Polymer Strengthening Layer 2 on top surface.

5. Optionally deposit Low Dissolution Rate Layer 3.

6. Temporarily hold silicon-on-insulator device or circuit with support material or holder (for example, via an electrostatic holder or polymer adhesion) to a quartz substrate followed by laser ablation to release.

7. Remove Handle Substrate (for example using plasma etching)

8. Optionally release from Electrostatic Holder

9. Deposit Low Dissolution Rate Layer 1 (surface optionally structured and or functionalized for improved cell adhesion)

10. Release from Electrostatic Holder or laser ablate surface of polymer adhesive laser by illuminating the surface of the polymer adhesive by laser ablation through the quartz substrate if not released in an earlier step.

11. Optional surfaces for functionalize for improved cell adhesion).

12. Dice.

### Particular Embodiments

One embodiment features two layers deposited by ALD: an inner electrical insulation layer of a material well suited for providing electrical insulation (for example, aluminum nitride) and a layer of a material well suited to serving as a low dissolution rate layer (for example, gallium nitride, titanium nitride, or titanium oxide) that might be electrically conducting. These two layers can be deposited one upon the other, in intimate contact with one another.

Certain embodiments have a rough outer surface than can improve the ability of an implant to integrate with a tissue without causing adverse reactions such as scarring. The device may have rounded corners and edges.

A device may have a low modulus similar to that of body tissue so that it is flexible.

It is possible to power and/or recharge devices using wireless power transfer including inductive charging or power transfer transmission of electromagnetic energy (for example radio frequency (RF) or light transmission).

In embodiments where the device is fully encapsulated by the low dissolution rate layer(s) (without an exposed electrode), non-electrical conduction contact sensing (including capacitive sensing, pressure sensing, and/or strain sensing, for example) can be used. These embodiments are also suitable for the above-described wireless power transfer.

Exemplary electrode devices are shown in FIGS. 1 and 2. The first coating layer and the second coating layer are in intimate contact in the region to the lateral sides of the circuit and in some embodiments the first low dissolution layer on the bottom side is in intimate contact with the second low dissolution rate layer on the topside and in the regions to lateral sides of the circuit. The implantable device can be designed to have a lateral dissolution distance greater than about 500 nm and in some embodiments the lateral dissolution distance can be more than 10 microns. A large lateral dissolution distance is desirable to increase the time for bodily fluids to dissolve through the low dissolution rate material to the electrical conductor. The implantable device can be designed to have a vertical dissolution distance greater than about 50 nm and in some embodiments the lateral dissolution distance can be more than 1 microns. A large vertical dissolution distance is desirable to increase the time for bodily fluids to dissolve through the low dissolution rate material to the electrical conductor.

Embodiments show in FIGS. 2 and 3 illustrate implantable devices device with an electrically conductive low dissolution rate material. A titanium nitride layer is an example of a low dissolution electrical conducting layer Titanium nitride is also a biocompatible material. Titanium oxide material has low levels of electrical conductivity and a low dissolution rate in in vivo environments. Titanium oxide has a dissolution rate or about 0.002 nm/day. Titanium metal is also electrically conductive. Titanium metal is considered the most biocompatible metal due to its resistance to corrosion in bodily fluids and bio-inertness, Titanium metal typically forms a thin titanium oxide layer on its surface in a bodily fluid environment. Titanium carbide has a low dissolution rate in bodily fluids. Titanium nitride, titanium oxide, titanium carbide, and titanium can be deposited by atomic layer deposition.

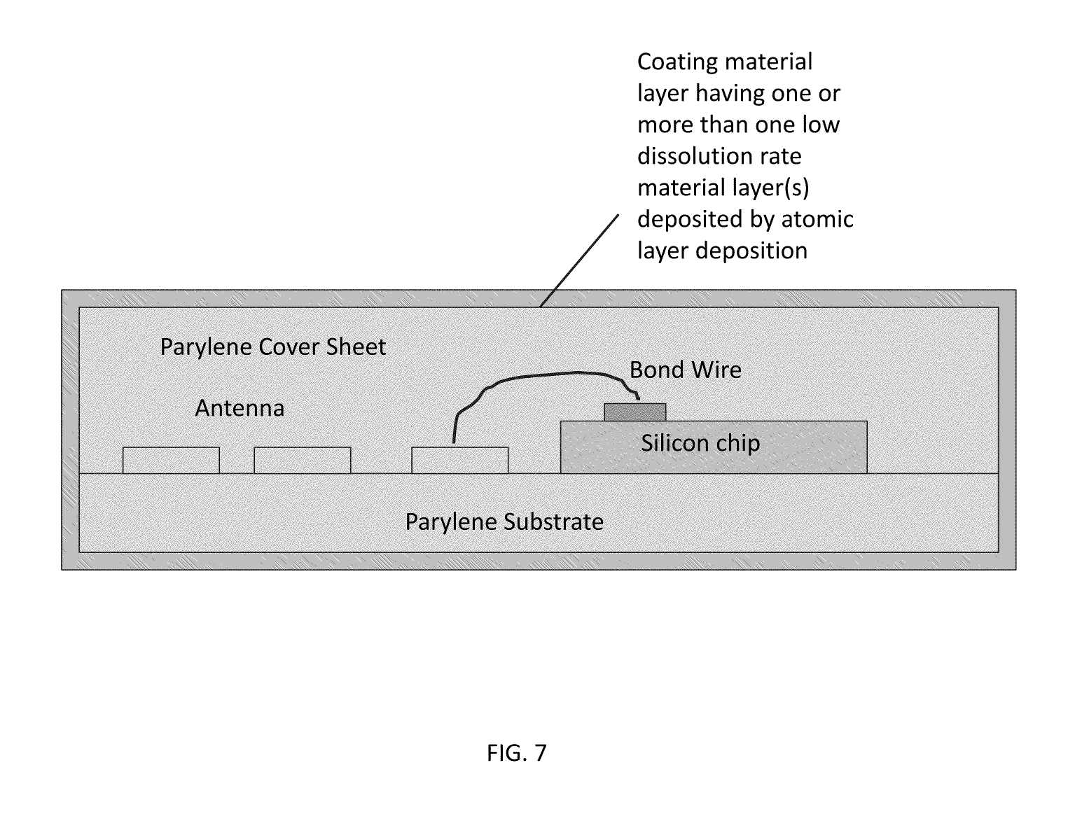

Certain embodiments, for example those illustrated in FIGS. 6 and 7, have a first ALD-deposited layer on the bottom side of an electrical conductor and a second ALD-deposited layer on the top side thereof. Embodiments of this sort can be obtained by the above-described First Prophetic Exemplary Process. A polymer on the top side strengthens and supports the structure so that it is stable under normal handling. The advantage of having the polymer on the top side rather than on the bottom side is that high temperatures can be used to deposit the ALD films. If the polymer was on the bottom side, then low temperature ALD deposition would be required.

In embodiments having elements connected by a bond wire, it is preferred that the bond wire is coated via ALD. Optionally, a polymeric coating (such as a parylene coating or parylene sheet) is then applied. For example, FIG. 9 shows an embodiment of a device where an ALD coating was applied on both sides of a substrate to protect a circuit including a bond wire, followed by parylene. The ALD coatings come together in an atomic interface to tightly seal the circuit.

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Anikeeva, P. “Biocompatible Materials for Optoelectronic Neural Probes: Challenges and Opportunities” The Bridge Vol. 43, No. 4, Winter 2013, pp. 39-48.
Greco, F. et al., “Patterned Free-Standing Conductive Nanofilms for Ultraconformable Circuits and Smart Interfaces,” ACS Applied Materials & Interfaces 2013 5 (19), 9461-9469.
Grill, W M. “Signal Considerations for Chronically Implanted Electrodes for Brain Interfacing.” in Indwelling Neural Implants: Strategies for Contending with the In Vivo Environment. Boca Raton (Fla.): CRC Press; 2008. Chapter 2
Pensabene, V. et al., “Flexible polymeric ultrathin film for mesenchymal stem cell differentiation,” Acta Biomaterialia, Volume 7, Issue 7, July 2011, Pages 2883-2891.
Ventrelli et al., “Influence of nanoparticle-embedded polymeric surfaces on cellular adhesion, proliferation, and differentiation.” J Biomed Mater Res Part A 2014: 102A: 2652-2661.
Xiang, Z. et al., “Ultra-thin flexible polyimide neural probe embedded in a dissolvable maltose-coated microneedle,” J. Micromech. Microeng. 24 (2014) 065015

What is claimed is:

1. An implantable device comprising:
a circuit protected with a low dissolution rate layer, wherein the circuit is either (a) fully encapsulated by the low dissolution rate layer and configured to perform non-electrical conduction contact sensing, or (b) partially encapsulated by the low dissolution rate layer with an electrode at least partially exposed outside the layer;
wherein the implantable device is suitable for implantation inside the body of a living animal; and
wherein the low dissolution rate layer comprises at least one element selected from the group consisting of gallium, boron, nitrogen, oxygen, zirconium, aluminum, and titanium.

2. The device of claim 1, wherein the low dissolution rate layer comprises a material selected from the group consisting of gallium, boron, nitride, oxide, zirconium, aluminum, titanium, gallium nitride, boron nitride, zirconium oxide, zirconia oxide, diamond, aluminum oxide, titanium nitride, titanium carbide, titanium dioxide, and combinations thereof.

3. The device of claim 1, wherein the device has shear modulus of 200 to 1500 Pa.

4. The device of claim 1, wherein said low dissolution rate layer has a dissolution rate of less than 3 nm/day at 96.4° C. while in an in vivo environment.

5. The device of claim 1, wherein said low dissolution rate layer is in a state of having been deposited by atomic layer deposition.

6. The device of claim 1, wherein said device is less than 25 microns thick.

7. The device of claim 1, further comprising an electrically insulating layer beneath said low dissolution rate layer, and wherein said low dissolution rate layer is electrically conductive.

8. The device of claim 7, wherein said low dissolution rate layer comprises GaN, TiN, and/or TiO₂ and is surrounded by an outer layer of AlN, each layer in a state of having been deposited by atomic layer deposition.

9. The device of claim 1, comprising circuit elements configured to perform wireless power transfer.

10. The device of claim 1, wherein said circuit includes at least one bond wire protected by said low dissolution rate layer.

11. The device of claim 1, wherein said low dissolution rate layer is surrounded by a polymeric coating.

12. The device of claim 1, wherein said polymeric coating is parylene.

13. The device of claim 1, having a rough outer surface.

14. An implantable device comprising:
a circuit protected with a coating material comprising two layers in a state of having been deposited by atomic layer deposition, namely an inner insulation layer and an outer low dissolution rate layer in intimate contact therewith,
wherein the circuit is either (a) fully encapsulated by both layers and configured to perform non-electrical conduction contact sensing, or (b) partially encapsulated by both layers with an electrode at least partially exposed outside the layers;
wherein the implantable device is suitable for implantation inside the body of a living animal; and
wherein the low dissolution rate layer is in a state of having been deposited by atomic layer deposition and comprises a material selected from the group consisting of gallium, boron, nitride, oxide, zirconium, aluminum, titanium, gallium nitride, boron nitride, zirconium oxide, zirconia oxygen, diamond, aluminum oxide, titanium nitride, titanium carbide, titanium dioxide, and combinations thereof.

15. The device of claim 14, wherein said low dissolution rate layer is gallium nitride and said insulation layer is aluminum nitride.

16. The device of claim 14, further comprising an outer layer of polymer.

17. A method of making an implantable device, the method comprising:
providing a substrate,
constructing a circuit on the substrate, and
coating, via atomic layer deposition, the circuit with a low dissolution rate layer comprising at least one an element selected from the group consisting of gallium, boron, nitrogen, oxide, zirconium, aluminum, and titanium
thereby obtaining an implantable device comprising a circuit protected with a low dissolution rate layer, wherein the circuit is either (a) fully encapsulated by the low dissolution rate layer and configured to perform non-electrical conduction contact sensing, or (b) partially encapsulated by the low dissolution rate layer with an electrode at least partially exposed outside the layer;
wherein the implantable device is suitable for implantation inside the body of a living animal.

18. The method of claim 18, wherein the substrate is a silicon-on-insulator substrate.

19. A method of making an implantable device, the method comprising:
providing a substrate,
providing a release layer on a substrate,
depositing a first coating material layer comprising at least one low dissolution rate material on the release layer,
constructing on the first coating material layer a circuit comprising an electrode material,
depositing on the circuit a second coating material layer comprising at least one low dissolution rate material such that both coating material layers contact each other at lateral sides of the circuit,
depositing a strengthening material layer on the second coating material layer,
etching a via through the strengthening material layer to the electrode material,
and etching the release layer to release the implantable device;
wherein the implantable device comprises the circuit protected with the low dissolution rate material, wherein the circuit is either (a) fully encapsulated by the low dissolution rate material and configured to perform non-electrical conduction contact sensing, or (b) partially encapsulated by the low dissolution rate layer with an electrode at least partially exposed outside the layer;
wherein the implantable device is suitable for implantation inside the body of a living animal; and
wherein the low dissolution rate material comprises at least one element selected from the group consisting of gallium, boron, nitrogen, oxygen, zirconium, aluminum, and titanium.

20. The method of claim 19, wherein said low dissolution rate material is electrically conductive, and further comprising depositing an insulating layer effective to electrically insulate said electrode material from said low dissolution rate material.

21. The method of claim 20, wherein said low dissolution rate material comprises GaN, TiN, and/or TiO₂

22. The method of claim 19, wherein both said coating materials are applied by atomic layer deposition.

23. The method of claim 21, wherein both said coating materials are applied by atomic layer deposition
